SEQUENCE LISTING (6 sequences)

SEQ ID NO: 1 - DNA, 18 bp, Phage M13
ggcgaaagggggatgtgc

SEQ ID NO: 2 - DNA, 22 bp, Phage M13
  modified_base (1)..(1): FAM fluorescent modification (green)
cggctcgtatgttgtgtggaat

SEQ ID NO: 3 - DNA, 25 bp, Chlamydia trachomatis
gggattcctgtaacaacaagtcagg

SEQ ID NO: 4 - DNA, 26 bp, Chlamydia trachomatis
  modified_base (1)..(1): ROX fluorescent modification (red)
cctcttccccagaacaataagaacac

SEQ ID NO: 5 - DNA, 25 bp, Chlamydia trachomatis
  modified_base (1)..(1): FMA fluorescent modification (green)
gggattcctgtaacaacaagtcagg

SEQ ID NO: 6 - DNA, 26 bp, Chlamydia trachomatis
cctcttccccagaacaataagaacac

(19) United States
(12) Patent Application Publication
(10) Pub. No.: US 2011/0065092 A1
(43) Pub. Date: Mar. 17, 2011

(54) USE OF NONVIABLE PARTICLES COMPRISING AN INTERNAL CONTROL (IC) NUCLEIC ACID

(75) Inventors: Einar Sverre BERG, Oslo (NO); Kjell SKAUG, Oslo (NO)

(73) Assignee: IC PARTICLES AS, Oslo (NO)

(21) Appl. No.: 12/879,324

(22) Filed: Sep. 10, 2010

Related U.S. Application Data
(63) Continuation of application No. 10/363,517, filed on Jul. 21, 2003, now abandoned.

(30) Foreign Application Priority Data
Aug. 30, 2000 (GB) GB0021303

(51) Int. Cl. C12Q 1/70; C12Q 1/68

(52) U.S. Cl. 435/5; 435/6

(57) ABSTRACT

The present invention relates to the use of non-viable particles (and in particular liposome particles, particles which are in the form of a viral protein coat, non-viable genetically modified organisms or particles made of synthetic polymers), comprising an internal control (IC) nucleic acid sequence as an internal control in nucleic acid-based analysis. The present invention further relates to non-viable particles comprising an IC nucleic acid and kits for carrying out the methods and uses of the invention.

### CROSS-REFERENCE TO RELATED APPLICATIONS

This application is a continuation of U.S. patent application Ser. No. 10/363,517, which is a §371 National Stage application of International Application No. PCT/GB01/03879, filed Aug. 30, 2001, which claims priority to Great Britain Application No. 0021303.3, filed Aug. 30, 2000, the disclosures of which are herein incorporated by reference in their entirety.

The present invention relates to the use of non-viable particles containing an internal control nucleic acid sequence in nucleic acid-based analysis. The present invention further relates to methods of nucleic acid-based analysis using these non-viable particles and kits for carrying out said methods.

The use of nucleic acid-based analysis has become extremely widespread during the last few years, particularly in the field of diagnostic testing. For example, such analysis has been used in the diagnosis of microbiological pathogens and genetic disorders and has also contributed to the discovery of unknown infectious agents and improved diagnostic tools. Such nucleic acid-based analysis can be either qualitative or quantitative and may or may not involve nucleic acid-based amplification techniques. The nucleic acid-based amplification assays, for example the polymerase chain reaction (PCR), ligase chain reaction (LCR), gap-filling LCR (Gap-LCR), nucleic acid sequence based amplification (NASBA) and transcription mediated amplification (TMA) have the advantages over more traditional microbiological tests of being very highly sensitive and specific. The major disadvantage of such assays and indeed other nucleic acid based analysis is however the obtaining of false positive and false negative results, which may in turn result in an erroneous interpretation of the data and inadequate or incorrect treatment of patients. In a worst case scenario, a false negative result might even result in a situation where a patient would be given the “all clear” for a particular disease and not be treated at all. The elimination or reduction of such false negative results is clearly desirable.

False positive results can be caused by contamination between different samples or by contamination of the sample with previously made amplification products, also called “carry-over” contamination. Much research and development has been undertaken to find ways to reduce the risk for “carry-over” contamination. Some commercial tests have incorporated quality assurance measures which use chemical procedures to reduce the “carry-over” contamination. The risk of obtaining false positive results is thereby virtually eliminated.

False negative results can be caused for example by the presence of inhibitory substances as impurities in the nucleic acid preparations. Many biological sample materials such as blood, saliva, urine and faeces contain such inhibitory substances that might interfere with the enzymes used in for example, amplification reactions causing partial or complete reduction of the enzyme activities or may cause the digestion or degradation of the nucleic acids to be tested. False negative results can also be due to erroneous execution of the assay procedure.

A current solution to try and minimise the number of false negative results which is used in both “in house” assays as well as in commercially available kits, involves the use of internal control (IC) nucleic acid sequences, or to which for example probes can bind in order to facilitate detection. Such IC sequences are designed for the particular assay in question and in general are nucleic acid sequences which contain regions to which particular primers can bind and initiate amplification of the IC sequence or to which for example probes or other entities can bind in order to facilitate detection. Such IC sequences can be so called “ideal”, “pseudo-ideal” or “non-ideal” IC sequences. An “ideal” IC sequence generally comprises a binding region, for example primer or probe binding regions which are substantially identical to the equivalent regions to which the primers or probes bind in the target nucleic acid to be detected. In addition, such “ideal” IC sequences generally have an identical sequence and are thus the same length as the target sequence. Thus, in this case the same primers or amplification probes can be used to amplify both the IC sequence and the target sequence and the efficiency of amplification should be the same in both cases. Alternatively, the use of probes (e.g. labelled probes) or other entities which bind to common sequences present in the IC and the target sequences similarly may provide quantitative information. However, for many applications “non-ideal” IC sequences can be used (see Cleland et al., Vox Sanguinis, Vol. 76: 170-174, 1999). Such “non-ideal” IC sequences generally comprise binding regions, for example primer or probe binding regions which do not bind to the primers which are used to amplify the target nucleic acid or probes which bind to such target nucleic acids. Assays involving such non-ideal IC sequences thus generally involve the use of a set of primers (or amplification probes) which can amplify the target sequence, together with a further set of primers (or amplification probes) which can amplify the IC nucleic acid. Alternatively such non-ideal IC sequences can contain a binding region, e.g. a probe or other entity binding region which is unique to the IC nucleic acid and not found in the target sequences.

In the case of nucleic acid-based assays which involve amplification, an amplification of the IC sequence in the absence of amplification of the target sequence will then be evidence of a correct negative result and the amplification of both sequences a correct positive result. In nucleic acid-based assays where amplification is not involved, detection may occur by binding a probe, and in which case the binding of a probe to the IC sequence in the absence of the binding of an appropriate probe to the target sequence will be evidence of a correct negative result and the binding of the respective probes to both the IC nucleic acid and the target sequence a correct or positive result.

Such IC sequences can have a variety of forms, e.g. they can be DNA molecules in the form of plasmids (Rosenstraus et al., J. Clin. Microbiol. 1998. vol. 36: 191-197) or RNA molecules (see WO93/23573 of New England Deaconess Hospital). In addition, in cases where the assay has been designed for the detection of a microorganism, a genetically modified organism has been used as an internal control (Kolk et al., J. Clin. Microbiology, 1994, vol. 32, 1354-1356).

An important feature of any IC sequence for use in nucleic acid-based assays is that it can be distinguished from the target sequence in the subsequent analysis or detection of the nucleic acid molecules produced. Methods of distinguishing IC sequences over target sequences often involve the design of an IC sequence so that it is a different size to the target sequences. Alternatively, the IC sequence can be engineered so that it contains a unique “probe binding region” that differentiates the IC from the target nucleic acid. In this way IC sequences (which may or may not have been amplified, depending on the assay in which they have been used) can be separated or distinguished from target sequences using an oligonucleotide to which the probe will interact. Such methods can be used in conjunction with “non-ideal” IC sequences as discussed above. However, the introduction of such distinguishing features into an “ideal” IC nucleic acid, where the IC has exactly the same sequence as the target sequence (i.e. the same primer or probe binding sites and the same sequence between the primer binding sites) would mean that strictly speaking such a modified IC sequence could no longer be regarded as an “ideal” IC sequence. In other words when the sequence of an “ideal” IC is modified, even if only by one base pair, such an IC is arguably no longer an “ideal” IC. Such IC nucleic acid sequences which can still bind to the same primers or probes as the target nucleic acid but contain modifications so that they can be distinguished from the target nucleic acid by methods such as those outlined above are referred to herein as “pseudo-ideal” IC sequences.

Advantageously, it has now been found that IC nucleic acids for use in nucleic acid-based assays can be encapsulated and contained in non-viable particles. Examples of non-viable particles which can be used in this way are liposomes, protein coats and non-viable genetically modified organisms.

The use of IC nucleic acids contained or encapsulated within non-viable particles in this way and in particular those contained within liposomes or protein coats have advantages over the use of viable genetically modified organisms, which have been used previously as internal controls. For example, such liposomes or protein coats containing IC nucleic acids are non-expensive to design and adapt to any nucleic acid amplification/detection system or any other non-amplification based nucleic acid assay and it is less labourious (and therefore less expensive) to make different kinds of liposomes or protein coats with regard to sequence and particle properties. In addition and perhaps most importantly, the non-viable particles in the form of liposomes, protein coats or non-viable genetically modified organisms are biologically safe, politically non-controversial, and contain no potential endogenous or exogenous hazardous sequences (e.g. antibiotic resistance genes).

The use of IC nucleic acids contained or encapsulated within such non-viable particles also has advantages over the use of naked DNA and RNA types of IC sequences discussed above. In this regard, it is important to realise that the idea behind an IC sequence is that it follows as precisely as possible as many as possible of the series of treatment steps that the target sequence undergoes. Most amplification based assays will comprise all or some of the steps of transportation of the sample to the analysis laboratory, storage prior to sample preparation, sample preparation (involving e.g. centrifugation or sedimentation), release and purification of nucleic acids, enzymatic amplification and detection of any amplification products. (Similarly non-amplification based assays will comprise all or some of those steps, with the exception of the amplification step). In the present “in house” and commercial assays that include an internal control, the internal control nucleic acid is added to the sample at the nucleic acid release/purification step or just before the amplification stage (see for example Rosenstraus et al. 1998, supra., which describes the technique behind the Roche commercial PCR assay). This has the effect that the steps which precede the addition of the IC have no quality assurance to ensure for example proper transportation and storage of the sample, efficient sample preparation (e.g. efficient centrifugation or sedimentation) and in some cases efficient release of nucleic acid. This is a serious drawback.

However, by using an IC nucleic acid which has been encapsulated in a non-viable particle in accordance with the present invention, the IC nucleic acid can preferentially be added to the samples at a very early time point in the process, i.e. such particles can even be added to the urine, blood etc. directly after the sample has been derived from the patient (i.e. at the collection step) and before transportation and/or subsequent processing to release nucleic acid takes place. (Of course the stage at which the particular type of non-viable particle is added to the sample will necessarily depend on the intention of the use of the internal control, e.g. as reference standard in quantitative analysis, as a quality control for the lysis of the cells in the sample, as a quality control for the detection step, etc). A conventional IC which was in the form of DNA or RNA which was not encapsulated would not necessarily be added at such an early time point as there is a significant risk that the samples will contain enzymes or other impurities (e.g. RNAase or DNAase enzymes) which will degrade the IC DNA/RNA while not degrading the target nucleic acid because it is not exposed to these elements (it is still within the cells present in the collected sample). In such a case, the IC sequence would fail to work as a quality control. The non-viable particles on the other hand would protect the IC nucleic acid at this early stage (like a cell membrane) with the IC nucleic acid only being released when the target nucleic acid was also released. In addition, it is not uncommon that the sample processing involves a centrifugation step or other steps for whole-cell-isolation/purification, meaning that any “naked” nucleic acid which had previously been added to the sample, would be discarded with the supernatant. This is another drawback of the use of a non-encapsulated IC. Thus, it can be seen that a non-viable particle encapsulated IC nucleic acid shows much improved properties over the prior art IC sequences and will, if required, allow the quality control of each step of the assay procedure.

Thus viewed from one aspect the present invention provides the use of non-viable particles comprising an internal control nucleic acid sequence as an internal control in nucleic acid-based analysis.

Viewed from another aspect the invention provides a method of nucleic acid-based analysis comprising the step of bringing the sample being analysed into contact with non-viable particles comprising an internal control (IC) nucleic acid sequence.

Generally and preferably the IC nucleic acid sequences are encapsulated within the non-viable particles.

“Encapsulated” or “encapsulation” as used herein in relation to the relationship between the IC nucleic acid and the non-viable particles refers to situations where all or part of the IC nucleic acid molecule is located/entrapped within the central core of the particle. Thus, the IC nucleic acids may be located entirely within the central core or pool of the particle, i.e. no part of said IC nucleic acid molecule is present at or on the external surface of said particle. Alternatively, all or part of the IC nucleic acid may be embedded or entrapped within or otherwise bound to the inner or internal surface of the particle. It is preferred that the IC nucleic acid (or at least a significant proportion of the IC nucleic acid) be encapsulated within the particles as described above as any nucleic acid located on the external surface of the particle will be at risk of degradation, e.g. by nuclease enzymes or contamination by inhibitory impurities which may be present in the sample. Thus, the terms “encapsulation” or “encapsulate” as used herein include any means or interaction between the non-viable particle and the IC nucleic acid, by which the non-viable particle protects the IC nucleic acid from the external environment surrounding the non-viable particle.

“Nucleic acid-based analysis or assays” as used herein refers to any analysis technique or assay, or one or more steps thereof, which is based on the quantitative or qualitative detection of nucleic acids. The main proviso is that the analysis technique has to be one which allows the use of an internal control sequence (in ELISA assays for example, the use of internal control sequences is not possible). Preferred nucleic acid based analysis techniques will be those which involve amplification of the nucleic acid in question (i.e. the target nucleic acid), e.g PCR, LCR, Gap-LCR, NASBA and TMA. In such assays it is also preferred that the IC nucleic acid be amplified alongside the target nucleic acid. There may be occasions however where the target nucleic acid is amplified in the nucleic acid based assay but the IC nucleic acid is not amplified (if for example a sufficient number of IC nucleic acid sequences are encapsulated within the non-viable particles such that amplification of said IC sequences is not necessary to obtain sufficient IC nucleic acid sequences to be detected, and/or for example the IC nucleic acid comprises PNA (peptide nucleic acid) which cannot be amplified). It will be appreciated however that in such cases the IC nucleic acid would act as a control for steps other than the amplification steps, i.e act as a control for all steps of the procedure in which it had been taken through the same process steps as the target sequences, e.g the detection steps.

It is important to note however that the nucleic acid-based analysis or assays discussed herein may not involve amplification of the target nucleic acid and/or IC nucleic acid. For example the assay may involve the binding of a probe or some other reagent to the target nucleic acid after which the binding of this entity can be detected. The branched DNA (bDNA) technique of Chiron might be mentioned in this regard (Urdea et al., 1991, Nucleic Acid Symposium Series, vol 24:197-200). This involves the hybridisation of a series of probes to a target nucleic acid. Each of these probes have a branched DNA attached which have signal moieties attached and which can give rise to an amplified signal. Thus, the probes can be detected in order to detect the target nucleic acid. Again in such assays it is preferred that the IC nucleic acid is also not amplified and is assayed in the same way as the target nucleic acid, e.g. by the binding of an entity to the IC nucleic acid and the subsequent detection thereof. If probes are used for example, then the IC nucleic acid may be designed to bind the same or different probe to the target nucleic acid. Either way the IC nucleic acid can still be used as an internal control.

“Internal control nucleic acid sequence” as used herein refers to any nucleic acid sequence which can function as an internal control in a nucleic acid based analysis. The IC nucleic acid can be any type of nucleic acid. For example it may be single stranded or double stranded DNA in a linear or circular form, for example in the form of a circular plasmid or a double stranded or single stranded oligonucleotide or PCR product. Alternatively, it may be RNA (for example sense or antisense RNA molecules or double stranded RNA molecules) or DNA/RNA hybrids. Alternatively, the IC nucleic acid may be PNA or a mixture or hybrid of PNA with other types of nucleic acid molecules. In some assays a mixture of different types of IC nucleic acid may be used e.g. a PNA IC may be used in addition to an RNA or DNA IC.

As is discussed further herein, depending on the type of nucleic acid-based assay being carried out, the IC nucleic acids comprise primer or probe binding sites or regions, or other sites or regions which can interact with appropriate assay reagents, such as capture probe hybridisation sites or probe detection sequences. Alternatively IC nucleic acids may carry or contain other distinctive information, e.g. have a particular length or detectable composition.

As outlined above the IC nucleic acid can be a “pseudo-ideal” IC nucleic acid sequence which may be amplified in essentially the same way and at the same rate as the target nucleic acid in the nucleic acid-based assay (or for nucleic acid based assays which do not involve amplification said pseudo-ideal IC nucleic acid is assayed or treated in essentially the same way as the target nucleic acid, e.g. is reacted and can bind to the same probe or entity as the target nucleic acid or the probes may be used after the assay for identification purposes only), but can be distinguished from said target nucleic acid by appropriate techniques. In this embodiment of the invention therefore, said IC nucleic acid can be amplified or e.g. probed using the same primers as are used to amplify the target nucleic acid or the same probes which are used to assay the target nucleic acid. In other words such IC nucleic acids comprise binding regions, e.g. primer or probe binding regions, or other sites or regions which can interact with appropriate assay reagents, such as capture probe hybridisation sites or probe detection sequences, or carry other distinctive information, e.g. have a particular length or detectable composition, which are capable of binding to the same primers or amplification probes as are used to amplify the target nucleic acid or are capable of binding to the same probes or entities which are used to assay the target nucleic acid, or have the same assessable informational content as the target nucleic acid. It will be appreciated that although such binding regions, e.g. primer or probe binding regions can be identical in sequence to the equivalent primer or probe binding regions in the target nucleic acid, complete identity is not absolutely necessary and what is required is that the binding regions, e.g. the primer or probe binding regions are substantially identical or sufficiently similar to the primer or probe binding regions of the target nucleic acid so that the primers or probes which bind to the primer or probe binding regions of the target nucleic acid can also bind to the primer or probe binding regions of the IC nucleic acid and function as primers, e.g. in extension reactions or, in the case of probes or other binding agents, function as entities in the nucleic acid based assay which can subsequently be detected. “Pseudo-ideal” IC nucleic acids with such binding regions, e.g. primer or probe binding regions are sometimes referred to herein as “near-ideal” IC nucleic acids.

In an alternative embodiment of the invention the IC nucleic acid can be a “non-ideal” IC nucleic acid. Such “non-ideal” IC nucleic acids generally comprise binding regions e.g. primer or probe binding regions, or other sites or regions which can interact with appropriate assay reagents, such as capture probe hybridisation sites or probe detection sequences, or carry other distinctive information, e.g. have a particular length or detectable composition, which are distinct/different from the primer or probe binding regions (or informational content) in the target nucleic acid and which cannot bind to the primers or probes (or do not have the same informational content) which are used to amplify or otherwise assay (e.g. by probe binding or assessing informational content) the target nucleic acid. Thus, in assays involving a non-ideal IC nucleic acid a different set of primers or probes or other entities is used to amplify or otherwise assay (e.g. by probe or other entity binding or recognition or assessment of informational content) the IC nucleic acid to the set of primers or probes which is used to amplify or otherwise assay the target nucleic acid.

Thus it can be seen that for the embodiments of the invention where a “pseudo-ideal” or “near-ideal” IC nucleic acid is used, where the nucleic acid-based assay involves amplification, the same set of primers (or amplification probes in the case of LCR) may be used to amplify both the target and the IC nucleic acid. In a PCR based amplification approach this set of primers will comprise a standard 2 primer PCR system (one primer designed to interact with the coding strand and the other with the complementary strand in the standard way). In a ligase chain reaction (LCR) based amplification system (e.g. LCR or Gap-LCR assays) amplification involves the use of 4 oligonucleotide amplification probes, 2 of which hybridise to the coding strand forming a nick between them and 2 of which hybridise to the complementary strand forming a nick between them. These two nicks are sealed by DNA ligase. In Gap LCR the pairs of amplification probes hybridise so that they are almost adjacent so that a DNA polymerase has to incorporate a few (1-3) nucleotides to fill the gap. Thereafter the ligase can seal the nick. This gives Gap-LCR a lower background since blunt end ligation of probes hybridised to its complementary probe can not occur. Thus, in the cases where “pseudo-ideal” or “near-ideal” IC nucleic acids are used, both the IC nucleic acid and the target nucleic acid will be amplified using the same four LCR amplification probes.

On the other hand, in the alternative embodiments of the invention where a “non-ideal” IC nucleic acid is used, in a PCR based amplification 4 primers will be used, 2 of which will interact with the coding and non-coding strands of the target nucleic acid to allow amplification thereof and 2 of which interact with the two complementary strands of the IC nucleic acid to allow amplification thereof. Non-ideal IC nucleic acids can also be used in amplification based assays based on the ligase chain reaction. Here four amplification probes will be used to amplify the target nucleic acid and two additional amplification probes will be used to amplify the IC nucleic acid. The two products will then be identical on one side of the nick position and different on the other side. If the IC is completely different from the wild type target, the reaction would need eight amplification probes, four amplification probes to amplify the target nucleic acid and four additional amplification probes to amplify the IC nucleic acid. Further information with regard to the design of appropriate IC nucleic acids for use in LCR based amplification systems (and in particular Gap-LCR) can be found in WO97/04128, the teaching of which is incorporated herein by reference.

As discussed further below an encapsulated, entrapped or embedded IC nucleic acid according to the present invention may also be used in the nucleic acid-based amplification reactions NASBA and TMA. These are both assays based on the use of two primers and thus, as for the standard PCR system as described above, where a pseudo-ideal IC nucleic acid is used only two primers are required, whereas for assays where a non-ideal IC nucleic acid is used four primers are required.

In general, for nucleic-based assays where quantitative results are required, it is necessary to use a “pseudo-ideal” or “near-ideal” rather than a “non-ideal” IC nucleic acid. On the other hand in assays where only qualitative results are required either a “pseudo-ideal”, “near-ideal” or “non-ideal” IC nucleic acid may be used.

Where the nucleic acid based assay involves amplification and the IC nucleic acid is designed to be amplified, the primers or amplification probes for inducing the amplification of the IC nucleic acid may optionally be encapsulated inside the non-viable particles with the IC nucleic acid. In such embodiments, amplification of the IC nucleic acid would of course not occur in the non-viable particles and would only occur once the non-viable particle was lysed and the IC nucleic acid and primers released into an appropriate environment for inducing amplification. Similarly in non-amplification based assays, the probes or other entities which are used to assay the IC nucleic acid may be encapsulated inside the non-viable particles with the IC nucleic acid.

A further important feature of the IC nucleic acid is that it can be distinguished from the target nucleic acid by any suitable method. Depending on the nucleic acid-based assay for which the IC nucleic acid is acting as a control, the IC nucleic acid may or may not have been amplified before said distinguishing takes place. Thus, if the nucleic acid based assay involves amplification then detection may take place straight away by any suitable technique (e.g. using a specific probe or on the basis of size, see below), or a further amplification of the IC nucleic acid and/or the target nucleic acid may take place before detection. Similarly, in nucleic acid-based assays which do not involve amplification, detection may take place directly or after an amplification step.

Methods of distinguishing IC nucleic acids from the target nucleic acid are well known and documented in the art (see for example the article by Zimmerman et al., Biotechniques, 1996 vol. 21: 268-279) and are discussed briefly above. A common method involves the distinguishing of the target and IC nucleic acid sequences on the basis of size. For example, the IC sequence might be designed so that it corresponds to the target sequence but contains a region of deletion or insertion located somewhere in the molecule, for example in between the primer binding regions which are used to amplify the sequence. This has the effect of allowing the target and the IC sequence to be amplified using the same primers but then enables the two species to be separated on the basis of their different sizes using for example any one of a variety of known chromatographic methods such as agarose gel electrophoresis, acrylamide gel electrophoresis, chromatographic size separation etc.

Distinguishing on the basis of size can also be used in assays involving a “non-ideal” IC nucleic acid. In such a case the IC nucleic acid is simply designed so that it is a different size to the target nucleic acid thus enabling the two to be distinguished by appropriate techniques. Alternative techniques which could be used to distinguish the target nucleic acid from the IC nucleic acid are well known and documented in the art and include the use of selective probe hybridisation or the use of primers, one or more of which have different labels incorporated therein and which thereby result in the IC nucleic acid and target nucleic acid (or amplification products thereof) being labelled differently.

An IC nucleic acid for use in the present invention can thus be designed for any assay and will generally be designed based on the particular target nucleic acid that is to be analysed and the nature of the nucleic acid-based assay to be used. The IC nucleic acid may (in the case of nucleic acid assays which involve amplification) be designed to contain sequences that can interact with the primers/LCR amplification probes in the amplification reaction mixture, often preferably with the same primers/amplification probes as the corresponding sequences in the target nucleic acid and may be further designed to contain a distinguishing feature such as an insertion or deletion region or a marker which will interact with a specific probe. Alternatively, the IC nucleic acid may be designed to contain sequences that can react with other entities which are used in the non-amplification based assay concerned, e.g. with probes, often preferably with the same probes/entities that bind to the corresponding sequences in the target nucleic acid. Again such IC nucleic acids may be further designed to contain a distinguishing feature (as described above).

Although in the case of a “pseudo-ideal” or “near ideal” IC nucleic acid it will be necessary to design the IC nucleic acid based on the sequence of the particular target nucleic acid that is to be analysed in order to ensure that the same binding reagents, e.g. primers or LCR amplification probes (in the case of an assay which involves amplification) or other probes or binding entities (in the case of assays which do not involve amplification) can be used to amplify or otherwise assay both the target and the IC nucleic acid, such “assay specific” IC nucleic acids are generally not required for the assays wherein a “non-ideal” IC nucleic acid is used. Thus, once designed a “non-ideal” IC nucleic acid could be used in any number of nucleic acid based assays for any number of different target nucleic acids provided the appropriate IC nucleic acid specific primer or probe set was introduced into the assay at the appropriate time and providing that the IC nucleic acid could be distinguished from the target nucleic acid. Such “non-ideal” IC nucleic acids are thus more general reagents.

Once designed, the construction of such IC nucleic acids can be carried out using techniques which are standard or conventional in the art, for example standard genetic engineering techniques (see the discussion in Zimmerman et al., supra). Furthermore, many examples of IC nucleic acids with such an appropriate design are well known and documented in the art and any of these IC nucleic acids may be used in the present invention. The document by Zimmerman et al., supra gives some examples of IC nucleic acids. Examples of “pseudo-ideal” IC's designed to be longer than the wild type target nucleic acid can be found in Ursi et al. (1992), APMIS 100(7): 635-9; Siebert and Larrick (1993), Biotechniques, 14(2): 244-9; Rosenstraus et al. (1998), Journal of Clinical Microbiology, 36(1): 191-7; Bretagne et al. (1993), Journal of Infectious Diseases, 168(6), 1585-8; Gilliland et al. (1990), PNAS USA, 87(7): 2725-9; Wang et al. (1989), PNAS USA, 86(24): 9717-21. Examples of “pseudo-ideal” ICs designed to be shorter than the wild type target can be found in Galea and Feinstein (1992), PCR Methods and applications, 2:66-69. Examples of “non-ideal” ICs can be found in Cleland et al. (1999) Vox Sanguinis 76(3):170-4; Rosenstraus et al. (1998), supra; WO 93/02215 and WO 97/04128. Ideally, in order for the IC nucleic acid to display similar kinetics, e.g. amplification kinetics, to the target nucleic acid, the IC nucleic acid is designed to be approximately the same length as the target nucleic acid. Thus, the length of the IC will generally be dependent on the length of the target nucleic acid. Preferably ICs are isolated nucleic acid molecules of less than 1000 bases. More preferably said ICs are less than 600 bases in length but greater than 40 bases in length, e.g. 50 to 500 bases or 100 to 500 bases in length.

The term “non-viable particle” as used herein refers to any entity which is capable of encapsulating, entrapping or embedding an internal control nucleic acid but which is not capable of propagation either alone (i.e. by self propagation) or by culture in a biological system which would normally allow the propagation of the entity in question. Such particles may never have been capable of being propagated, e.g. liposomes, protein particles, or synthetic particles or other particles which consist solely of an encapsulating shell and do not contain any genetic material which enables replication and propagation of the particle in a biological system, for example particles which are made up of viral coat proteins or viral capsid proteins. Other non-viable particles included herein are those which were capable of being propagated, e.g. virus particles or other pathogenic organisms, but which have been altered in such a way that replication and/or propagation of the particles are no longer possible. For example such particles could include genetically modified organisms (GMOs) which have been further modified so that they are no longer viable in terms of propagation and/or replication, described herein as “dead” or non-viable GMOs.

Such particles may thus be made of any material which is capable of encapsulating, entrapping or embedding a nucleic acid. Such material may for example include lipids or modified lipids (e.g. as part of a liposome or liposome type particle) or may include proteins (e.g. in the form of a protein coat such as the protein coat or “capsid” of a virus) or a combination of lipid and protein (e.g. where proteins are embedded in a lipid vesicle in a way which will mimic the normal protein embedded lipid bilayer of a cell). Such particles may also be made of synthetic material, e.g. a synthetic polymer. Exemplary synthetic materials/polymers for use in the encapsulation of IC nucleic acids in accordance with the present invention include cationic polymers such as those described in Wolfert et al., 1999, Bioconjugate Chemistry, 10: 993-1004, which entrap nucleic acids in a cationic polymer-nucleic acid complex and polylysine based complexes such as those described in Wagner et al., 1992, PNAS USA, 89: 7934-7938.

Another important feature of the “non-viable particles” which encapsulate, entrap or embed the IC nucleic acid is that the structure of the particles will lyse, collapse, leak, i.e. generally be disrupted, under the same conditions which will “disrupt” the target entity e.g. cells or viruses which contain the target nucleic acid which is being analysed. Whilst preferably the target nucleic acid is contained within cells or viruses and subsequent discussion refers to such target cells or viruses, the methods of the invention may be used in assays in which the target nucleic acid is contained within an entity other than a cell or virus, e.g. a non-naturally occurring particulate structure, such as a liposome. Target “cells” include cells derived from multicellular organisms or unicellular organisms (e.g. yeast, protozoa and bacteria). Target “viruses” include any viruses, including bacteriophage. The term “disrupt” as used herein thus includes any disruption (e.g. lysis etc. as mentioned above) which will result in release of the contents of the non-viable particle/cell/virus, i.e. the release of at least the IC molecule (from the non-viable particle) and the target nucleic acid (from the target entity e.g. target cell or virus). Such conditions generally involve the exposure of the target cells or viruses to appropriate lysis reagents which often contain detergents, and/or reagents such as strong acids, strong bases or organic compounds such as phenol or guanidine thiocyanate. Preferably such particles will also be of the same or similar stability under assay and sample conditions (such as temperature, salt concentration, etc.) as the target cell or virus. The ability of the non-viable encapsulating, entrapping or embedding particle to “mimic” as far as possible the target cell is a central theme of the present invention and generally any modification which can be made to the non-viable particles in order that they more closely resemble the target cells or viruses which contain, e.g. encapsulates the target nucleic acid is an advantage and such modified particles are included in the above definition. For this reason, for some applications particularly preferred particles are liposome particles or protein particles in which all or some of the lipids or proteins have been modified (e.g. so that they do not correspond to naturally occurring or native proteins or lipids) so that they exhibit an advantageous property such as increased stability. Known modifications which can be used to increase the stability of liposomes for example are discussed further below.

Additionally or alternatively the non-viable particles can be designed to include proteins which are naturally found in the membrane of the target cells. The inclusion of such proteins will enable the particles to more closely mimic the target cell but may also be used in order to target the delivery of a liposome.

Preferred non-viable particles for use in the present invention are liposome particles, particles which are in the form of a viral protein coat, “dead”/non-viable GMOs or particles made of synthetic polymers. More preferred non-viable particles are liposome particles, “dead”/non-viable GMOs or particles made of synthetic polymers. Other preferred non-viable particles are those comprising modified proteins or lipids as described above. Most preferred non-viable particles are liposome particles.

As mentioned above it is important that the non-viable particles used for a particular assay mimic as far as possible the target cells or viruses which contain the target nucleic acid. Thus, the appropriate encapsulation (or entrapment, embedding) vehicle, e.g. a liposome, a viral protein coat, a “dead” GMO or a synthetic particle as described above, for a particular assay will be selected depending on the nature and characteristics of the target cell or virus and also the conditions under which the target cells or viruses will lyse. The appropriate encapsulation, entrapment or embedding vehicle will be one which will allow the encapsulated, entrapped or embedded IC nucleic acid to undergo the same treatment steps as the target cell or virus and target nucleic acid under assay. Thus the appropriate encapsulation, entrapment or embedding vehicle will for example be one which can be separated from the sample with the target cells in the same step (e.g. by centrifugation or sedimentation) and which can be lysed, disrupted etc. under the same conditions which lyse the target cells or viruses.

As the separation step is often carried out by centrifugation or sedimentation, in order to ensure that the non-viable particles can be separated from the sample with the target cells or viruses, it is often useful to select non-viable particles with similar weights and/or densities as the target cells or viruses. Methods for altering the density/weight of liposomes are well known and documented in the art. For example the liposome density can be increased by filling the central core of the liposome with a more dense solution, such as cesium chloride (or other heavy compounds) or a high salt solution. Alternatively and preferably, density of the liposomes can be increased by the incorporation of polysaccharides, for example Blue Dextran or Dextran sulphate into the liposomes. Said polysaccharides may be incorporated into the central core and/or the lipid membrane of the liposome. “Dead” GMOs which are produced by modifying the live GMO so that it can no longer replicate and propagate should automatically have the same density as the target entities, e.g. living target cells. The viral protein particles will also have practically the same density as the living target cells. A way of making such particles has been described by Pear et al., (1993), PNAS USA 90: 8392-8396.

If the sample preparation to obtain the target nucleic acid from the target cell or virus includes a step of selective cell capture, for example an immunoseparation step or other type of affinity separation step to bind cell surface proteins (or other molecules), then such surface proteins or molecules may need to be introduced into or onto the non-viable particles in order that the non-viable particles can be separated by the same steps as the target cells. The introduction of proteins and other markers into the lipid membrane of a liposome particle can be carried out using techniques which are documented in the art (see for example Rongen et al., 1997, J. of Immunol. Methods, 204(2): 105-33) and thus when a selective cell capture step is involved, appropriate liposome particles can be adapted for such use. The dead GMOs and the protein coats will often automatically have the particular marker entrapped on the surface. This is particularly the case where said protein coats or dead GMOs are prepared based on, e.g. by modifying, the target cells in question. For example where the target cell is a type of microorganism then an appropriate encapsulation, entrapment or embedding vehicle would be an equivalent “dead” microorganism which would automatically express the same marker proteins on its surface.

Generally, the composition of the non-viable particles should be as simple as possible. Thus, if an unmodified simple non-viable particle has a similar stability to the target cells, can be separated with the target cells in the same step and can be lysed with the target cells under the same conditions, then none of the above discussed modifications such as the inclusion of proteins in the particle surface should be necessary or desired. For example, in an assay for the microorganism Chlamydia trachomatis a simple centrifugation step can be used to separate the cells of the microorganism from the sample and thus a simple liposome (or other simple non-viable particle) with no modifications (such as proteins in the lipid membrane) but which has a density greater than the density of the fluid making up the sample (so that it can be centrifuged to the bottom of a vessel with the Chlamydia) and which will lyse under the same conditions as Chlamydia will be suitable for encapsulating, entrapping or embedding an IC nucleic acid.

The term “liposome particle” as used herein includes all types of liposomes and liposome-type vesicles known in the art. Thus, at its most general, a “liposome particle” may be any lipid-based vesicular structure. The IC nucleic acid once designed and produced as described above can be introduced into liposome particles by methods which are well known and standard in the art. In this regard, the nucleic acid might either be encapsulated within the internal aqueous pool or core of the liposome and/or be bound to the inner surface of the liposome, for example by electrostatic forces or entrapped or embedded within the vesicular forming lipid-based layer, preferably within the inner surface. In the methods and uses of the present invention it is preferred that the nucleic acid be encapsulated within the liposome (or other type of non-viable particle) and/or be bound to or entrapped or embedded within the inner surface of the liposome (or other type of non-viable particle), as any nucleic acid on the external surface of the particle will be at risk of degradation by nuclease enzymes or contamination by inhibitory impurities which may be present in the sample.

Any method of encapsulation of the nucleic acid may be used. However, there are three main methods described in the literature for the encapsulation of nucleic acids, (i) the reverse phase evaporation method, (ii) the dehydration/rehydration method and (iii) freeze/thawing (F. Szoka Jr., et al., Proc. Natl. Acad. Sci. USA 75 (1978) 4194-4198; D. W. Deamer, et al., J. Mol. Evol. 18 (1982), 203-206; U. Pick, Arch. Biochem. Biophys. 212 (1981), 186-194; M. J. Hope, et al. Biochim. Biophys. Acta 812 (1985) 55-65; C. J. Chapman et al, Chem. Phys. Lipids 55 (1990) 73-83; and Monnard et al., Biochim. Biophys. Acta 1329 (1997) 39-50) and the use of one of these methods is preferred. Indeed the freeze/thawing method has been shown to be particularly efficient in the encapsulation of nucleic acids (Monnard et al., supra) and this method is preferred.

The efficiency of entrapment/encapsulation tends to vary depending on the conditions used during the encapsulation process and the lipid composition of the liposomes themselves. Appropriate encapsulation conditions for a particular IC nucleic acid and liposome formulation can of course be derived by routine trial and error. Some preferred liposome formulations for use in the present invention are discussed further below. In general, the efficiency of entrapment appears to be enhanced if the method of preparing the liposomes and encapsulation of the IC nucleic acid involves extrusion of the liposome dispersion, for example by forcing the dispersion through one or more filters with appropriate pore sizes. Liposomes which have been prepared using extrusion are thus preferred for aspects of the invention where high encapsulation efficiency is desired. Methods of extrusion and the selection of appropriate pore sizes are well known and documented in the art.

In general, the size of the IC nucleic acid which is to be encapsulated or embedded into or entrapped in the liposome does not represent a problem, with small nucleic acid fragments (e.g. fragments of the order of tens of base pairs, e.g. fragments of 10 to 100 base pairs and fragments of the order of a few hundred base pairs) and larger molecules (of the order of a few kilobases) being encapsulated with similar efficiency. Again however adjusting the conditions may be used to improve encapsulation, embedding or entrapment if it is not adequate.

Other methods may be used to facilitate uptake of nucleic acids into liposomes. For example a nucleic acid in aqueous solution might be used to hydrate a dried mixture of lipids making up the liposomes, in which case the nucleic acid will be incorporated into the fluid filled core of the liposomes during liposome formation. Alternatively methods wherein cationic lipid-DNA complexes can be made to form spontaneously when lipids are mixed with DNA can be used to facilitate the entrapment of the nucleic acids into liposomes (see for example Felgner et al., 1987, PNAS USA, 84: 7413-7417 and Hofland et al., 1996, PNAS USA, 93, 7305-7309).

Liposomes are microscopic spherical particles in which membranes, consisting of one or more lipid bilayers, encapsulate a fraction of the solvent in which they are suspended into their interior. Liposomes and methods of preparation thereof are well known and documented in the art. Thus, the properties of the liposome particles into which the IC nucleic acid is to be introduced can be manipulated and selected by methods well known and documented in the art. Liposomes are generally composed of phospho(lipids) and may be composed of one or a mixture of lipids with varying properties. Modified lipids, such as lipids modified with polyethylene glycol can also be used, as can liposomes coated with inert hydrophilic polymers (so-called “stealth” liposomes, Lasic, 1995, CRC Press) or liposomes which have been modified to include stabilising proteins such as S-layer protein (Mader et al., 1999, Biochimica et Biophysica Acta. 1418: 106-116).

The appropriate amount of each type of lipid to be incorporated into the liposomes will be selected based on the particular use to which the liposomes will be put. The individual lipids making up the liposomes can have an overall positive, negative or neutral charge. Thus, depending on the particular combination and quantities of lipids used, the liposome particles themselves can have an overall positive (cationic), negative (anionic), or neutral charge.

Preferred liposomes for the most efficient encapsulation, embedding or entrapment of nucleic acids will comprise an overall positive charge, i.e. will be “cationic” liposomes containing a proportion of positively charged lipids. Examples of such cationic (positively charged) lipids are DOTAP (1,2-dioleoyloxy-3-(trimethylammonium)propane), DOGS (N,N-dioctadecylamidoglycylspermine), DDAB (dimethyldioctadecylammonium bromide), DOTMA (N-[1-(2,3-dioleyloxy)propyl]-N,N,N-trimethylammonium chloride), DOSPA (2,3-dioleyloxy-N-[2(spermine-carboxamido)ethyl]-N,N-dimethyl-1-propanaminiumtrifluoroacetate) and DMRIE (N-[1-(2,3-dimyristyloxy)propyl]-N,N-dimethyl-N-(2-hydroxyethyl)ammonium bromide). Particularly preferred liposomes for use in the present invention comprise one or more neutral lipids such as POPC (1-palmitoyl-2-oleoyl-sn-glycero-3-phosphocholine) or DOPE (1,2-dioleoyl-3-sn-phosphatidylethanolamine), and one or more of the positively charged lipids DDAB, DOTAP or DOSPA. The particular proportions of lipid which are optimum for encapsulation, embedding or entrapment can be determined by routine trial and error, but an exemplary liposome composition might comprise from between 1% to 50% positively charged lipid, for example from between 1% to 10% DDAB (or another positively charged lipid) or from between 20% to 50% DOTAP (or another positively charged lipid), or a ratio of positively charged lipid to neutral lipid of from approximately 0.5:1 to approximately 5:1, for example a ratio of DOSPA (or another positively charged lipid) to a neutral lipid (e.g. DOPE) of approximately 3:1 or a ratio of DOTAP (or another positively charged lipid) to a neutral lipid (e.g. DOPE) of approximately 1:1.

A further type of particularly preferred liposomes are those which comprise a proportion of phospholipid which are phospholipid derivatives of polyethylene glycol, for example PEG-PE (N-(ω-methoxypoly-(oxyethylene)oxycarbonyl)-DSPE). (DSPE=1,2-distearoyl-3-sn-phosphatidylethanolamine). Such liposomes are also preferably cationic liposomes.

A yet further type of preferred liposomes are those in which a polysaccharide (e.g. Blue Dextran or Dextran sulphate) has been incorporated, for example to increase the density of the liposomes. Said polysaccharide can be incorporated into the central core and/or the lipid membrane of the liposome.

Most particularly preferred liposomes comprise the lipids POPC and DDAB, preferably in a ratio of 97.5:2.5, or the lipids DOTAP, DOPE and PEG-PE, preferably in a molar ratio of 25:25:3. Optionally, these liposomes may also comprise polysaccharides such as Blue Dextran or Dextran sulphate.

Other examples of lipids that can be used to form the liposome molecules in accordance with the present invention are well known and documented in the art. Some examples of neutral lipids include cholesterol, DPPC (dipalmitoylphosphatidylcholine), egg phosphatidylcholine and soybean phosphatidylcholine. Examples of negatively charged lipids include PS (phosphatidyl serine), PI (phosphatidyl inositol), ganglioside GM1, PG (phosphatidyl glycerol) and PA (phosphatidic acid). Further examples of positive lipids include HDA (hexadecylamine).

Liposomes can be produced in various sizes from small (often unilamellar) vesicles of 50-150 nm to large (often multilamellar) vesicles of a few μms. The size range can be chosen as appropriate and is a compromise between loading efficiency of liposomes (increases with increasing size) and liposome stability (decreases with increasing size above an optimal 80-200 nm range). The choice of liposome size can be determined by routine trial and error. However, a preferred size for the liposomes might be in the range of 80-200 nm.

Preferred liposomes for use in accordance with the present invention are liposomes which are stable in biological media and fluids such as for example blood, serum, faeces, urine etc. The stability of liposomes can be increased by for example coating liposomes with inert hydrophilic polymers such as PEG or by coating them with proteins derived from thermally stable bacteria (for example coating with S-layer protein—see Mader et al., Bioch. Biophys. Acta. 1418 (1999) 106-116). Alternatively the use of lipids derived from thermophilic bacteria could be considered (see e.g. Chang. Bioch. Biophys. Research Communications, vol. 202, 1994, 673-679). As discussed above the stability of the liposome is designed to mimic the stability of the target cells.

Once prepared the liposomes can either be used immediately in the methods and uses of the present invention or stored for future use. If the liposomes are to be used immediately, conveniently they are used in the form of an aqueous suspension. If the liposomes are to be kept for an extended period of time before use then preferably the preparations will be freeze dried or lyophilised for storage using methods well known and documented in the art. Such dried preparations can then be rehydrated for use at the appropriate time.

For the embodiment where the IC nucleic acid is encapsulated within or embedded or entrapped in a viral protein coat, the particular IC nucleic acid can be encapsulated using methods which are well known and documented in the art. Such methods will generally involve the mixture of the various proteins making up the protein coat in appropriate portions, together with the particular IC nucleic acid which is to be encapsulated and subjecting said mixture to conditions which induce the assembly of the viral proteins to form a viral coat particle. In this way the IC nucleic acid will automatically become trapped/encapsulated within the viral coat particle. Assembly of virus capsids might for example be carried out in vitro, either after e.g. disruption of the capsid by chelating agents and reassembly by addition of calcium (e.g. Brady et al. 1979, J. Virol. 32: 640-647, Paintsil et al. 1998, J. General Virol. 79: 1133-1141) or after in vivo expression of the capsid proteins (Sternberg 1990, PNAS USA, 87: 103-7; Hwang et al. 1994, PNAS USA, 91: 9067-71; Tellinghuisen et al. 1999, J. Virol, 73: 5309-19).

For the embodiments where the IC nucleic acid is encapsulated in a “dead” or non-viable GMO, the IC nucleic acid could first be cloned into the appropriate GMO using standard and well known techniques, after which the GMO could be inactivated or “killed” in such a way that the cell surface/cell membrane of the GMO and the IC nucleic acid remained unaffected or intact, thereby leaving the IC nucleic acid trapped inside the cell membrane of a non-viable GMO. Suitable reagents which could be used to inactivate the genetic and cellular machinery of the GMO without affecting the cell membrane or the IC nucleic acid are well known in the art and include for example antibiotics, antimicrobial agents or other agents that target and disrupt protein synthesis, for example by disrupting or altering ribosome functions. Examples of antibiotic or antimicrobial agents that could be used to inactivate the GMOs are tetracyclin which inhibits protein synthesis, rifamycin that inhibits transcription and nalidixic acid that inhibits DNA gyrase and blocks replication (Davies and Smith 1978, Annual Review of Microbiology, 32: 469-518).

Although it is known to introduce nucleic acids into non-viable particles such as those discussed above, it is nowhere disclosed in the prior art that the nucleic acid to be introduced can be an IC nucleic acid. Thus, non-viable particles, for example liposome particles, synthetic particles, viral coat protein particles or “dead” GMOs comprising an IC nucleic acid form further embodiments of the present invention. Non-viable particles such as those described herein for use in the methods and uses of the invention described herein form yet further aspects of the invention.

Especially preferred nucleic acid-based assays in which the IC nucleic acids are used are those in which there is a risk of false negative results occurring, for example in diagnostic assays, especially those carried out on samples derived directly from the patient. Alternatively or additionally, the liposomes (or other non-viable particles) and internal controls of the present invention may be used simply to monitor the amplification of nucleic acids in any qualitative nucleic acid based test, for example in a PCR or LCR based test. “Pseudo-ideal” or “near-ideal” or “non-ideal” IC nucleic acids can be used for qualitative nucleic acid based tests.

Furthermore, as will be described in more detail below, whatever the source of the sample, the non-viable particles and the IC nucleic acids of the present invention can be used in any assay where quantitation of the nucleic acids produced is required. Where quantitation is required, the use of a “pseudo-ideal” or “near-ideal” IC nucleic acid is necessary whereby the IC nucleic acid and the target nucleic acid are amplified and/or detected or otherwise assayed by the same probes or primers (or informational content, etc).

A preferred method of nucleic acid-based analysis according to the present invention will comprise the steps of:

(i) obtaining a sample to be analysed;

(ii) bringing said sample into contact with non-viable particles comprising an appropriate internal control nucleic acid;

(iii) inducing the release of the nucleic acid to be analysed from within the sample and the release of the internal control nucleic acid from within the non-viable particles; and

(iv) analysing the released nucleic acids.

In step (i) the sample to be analysed may be any sample on which it is desired to carry out a qualitative or quantitative nucleic acid-based assay.

Said samples may thus be derived from in vitro sources (such as cultured cells, bacteria or viral particles) or in vivo sources (such as samples derived from human, plant or animal sources) or synthesized in the case of samples containing non-naturally occurring target entities. Other samples might be those that are tested for detection of food pathogens. If the sample is derived from a patient or an animal, the appropriate type of biological sample to obtain will vary depending on the nature and source of the nucleic acid being analysed, but examples include blood, serum, plasma, saliva, faeces, urine, milk and organ, tissue or cellular extracts or secretions, e.g. mucosal secretions etc. Such samples are likely to be fairly impure and contain all manner of enzymes and other compounds which will inhibit or impair the enzymes involved in DNA amplification (or inhibit or impair other enzymes or entities involved in non-amplification based nucleic acid assays), or will degrade any nucleic acid (e.g. the target nucleic acid or any IC nucleic acid) which is itself present. Methods for obtaining appropriate samples from patients or animals are well known and described in the art.

The addition of the non-viable particles to the sample (i.e. step (ii)) can be carried out in any convenient way. The sample to be analysed is likely to be in the form of a solution or can conveniently be made in the form of a solution e.g. by adding fluid and/or tissue disrupting or dissolving agents. Appropriate preparations and properties of non-viable particles for use in accordance with the present invention are defined and described above. Thus, the non-viable particles when bought into contact with the sample may be in the form of an aqueous suspension of said particles or may be in a dried form which are then rehydrated and reconstituted when added to the fluid sample.

In a preferred embodiment of the invention the time between step (i) (obtaining the sample) and step (ii) adding the non-viable particles is kept to a minimum. Thus, the particles are added to the samples as soon as practicable and ideally are added immediately after sample is obtained in vitro or collected from the patient or animal. It should be borne in mind however, that for the methods of the invention to work, the non-viable particles can be added to the sample at any convenient time before, at the same time, or during the time that the inducing of the release of the nucleic acid from the sample (i.e. step (iii)) takes place. The particles should not be added after this time because then the IC nucleic acid will not be released from the particles at the same time point that the target nucleic acid is released from the target cells and therefore will not be taken through the procedure with the target nucleic acid.

Step (iii) of the above discussed method involves inducing the release of the nucleic acid to be analysed from within the sample and the IC nucleic acid from within the non-viable particles. Conveniently the nucleic acids from the sample (e.g. from the cells or viruses of the sample) and the particles are jointly released, i.e. are released at the same time and under the same conditions. Accordingly, an appropriate lysis buffer or other disruptive agent or conditions which induce the disruption of both the membranes of the target cells (or other target entities) and the structure of the non-viable particles used in the assay should be selected. This is of course not difficult to do when the non-viable particles are liposomes as the nucleic acid from the sample will normally be contained in cells and the liposomes by their very nature resemble cell membranes. Thus, conditions (for example lysis buffers), which will disrupt/lyse the membranes of the cells or other target entities in the sample and release the nucleic acid contained therein, should in general also disrupt/lyse the liposome particles. Similar considerations apply when the non-viable particles used are “dead” GMOs. “Dead” GMOs will generally be appropriate non-viable particles in assays where the target cells are the equivalent or similar “live” wild type organisms and thus again it is clear that conditions under which the wild type cells are lysed will also result in lysis of the “dead” GMOs.

Appropriate lysis medium/buffers are well known and standard in the art and may for example contain detergents or proteinases or protein denaturing agents such as guanidine thiocyanate which will disrupt the membranes of the target cells or viruses (and the liposome membranes) and also the particles which comprise a viral protein coat. Other standard ingredients such as protease inhibitors, DNase and RNase inhibitors, preservatives, chelating agents etc. can also be added to the lysis buffer if desired or necessary.

It will be usual practice that before inducing the release of the nucleic acids in step (iii) the cells (or other target entities) and particles will be separated from the other materials in the samples. Such separation can conveniently be carried out for example by centrifugation of the target cells or viruses and liposomes (non-viable particles) into a pellet and carefully removing the unwanted supernatant before the lysis medium is added. Thus, such a separation step is an optional step in the above discussed methods.

As the samples are generally obtained from patients the above method may optionally involve the steps of transporting the sample to the analysis laboratory and storage of the sample prior to sample preparation. Such transportation and/or storage steps might occur after step (i), step (ii) or step (iii).

Once the nucleic acids have been released these can be analysed (step iv). Generally said analysis step will involve carrying out an appropriate nucleic acid-based analysis or assay as described above, which may or may not involve amplification, followed by some kind of examination or detection step. Analysis may be carried out on the crude cell (and non-viable particle) lysate directly (generally after amplification of the nucleic acids to be analysed has been carried out, see below), or on DNA or RNA or PNA molecules which have been further purified from the crude cell (and non-viable particle) lysate by standard methods. In general, if the nucleic acid which is being analysed is a DNA molecule, then the analysis of the crude cell (and non-viable particle) lysate is not problematic and the same reproducibility can be obtained as with purified DNA. Moreover, the use of crude cell lysates entails less labour and avoids the potential loss of specific sample which may occur during the DNA purification. Where the nucleic acid to be analysed is RNA, generally a further purification step to isolate the RNA from the crude lysate is preferred.

Usually, in order to aid analyses of the nucleic acids an amplification step will be carried out at some stage. Amplification will normally be required as the amount of target nucleic acid present and/or IC nucleic acid present in a sample is likely to be at a concentration below that which is easily detectable. Thus, if the nucleic acid-based assay involves amplification, this amplification may be followed by a further amplification step before examination or detection occurs. Alternatively, if the nucleic acid-based assay does not involve amplification then an amplification step may be carried out before examination or detection of the nucleic acid occurs. Alternatively, no amplification step may be required before examination or detection takes place.

Amplification can be carried out by any of the techniques well known and documented in the art, e.g. PCR, LCR, Gap LCR, NASBA or TMA. For the DNA based amplification reactions to occur a DNA rather than an RNA molecule should be present as a template. Thus, it should be borne in mind that if the IC nucleic acid and/or target nucleic acid are RNA molecules then these should first be reverse transcribed into DNA molecules which can then be amplified by the usual techniques. As discussed above, the primers or amplification probes used for such amplification may be specific for either the IC nucleic acid or the target nucleic acid or may be designed so that they amplify not only the target nucleic acid (if present) but also the IC nucleic acid.

Regardless of whether the analysis step involves amplification, where that is performed, the examination and detection part of the analysis step will generally depend on the distinguishing feature of the IC nucleic acid over the target nucleic acid. If for example the IC nucleic acid is of a different size to the target then the analysis can conveniently be carried out by separating the nucleic acids on an agarose gel and visualising the nucleic acids in the sample by e.g. ethidium bromide staining or by e.g. separating the nucleic acids by capillary gel electrophoresis and detecting the nucleic acids in the sample by virtue of some kind of incorporated label, e.g. a fluorescent label incorporated into one or more of the primers or probes used. In embodiments of the invention where different sets of primers or probes, or other entities are used to analyse the target nucleic acid and the IC nucleic acid (i.e. in embodiments where non-ideal IC nucleic acid sequences are used), preferably different labels, e.g. different fluorescent labels, may be incorporated into one or more of the primers or amplification probes of each primer or amplification probe set so that the amplification products deriving from the IC nucleic acid or the target nucleic acid (if present) can be distinguished using appropriate apparatus and/or software. Alternatively, the amplification reaction may be carried out using a proportion of labelled nucleotides (e.g. fluorescent or radiolabelled nucleotides), in which case the separated and labelled nucleic acids can be visualised by appropriate detection means well known in the art. In methods of nucleic acid-based analysis which do not involve amplification the probes or entities which may be used to assay the IC nucleic acid and/or the target nucleic acid can also be engineered to carry appropriate labels other entities which can be detected and distinguished. Alternatively, in such non-amplification based methods it may be possible to distinguish the target nucleic acids from the IC nucleic acids by other methods, e.g. by virtue of size or other informational content.

The analysis of the nucleic acids in step (iv) can either be qualitative e.g. an observation as to whether the band corresponding to the target nucleic acid is present or absent, or may be quantitative in that the concentration of the target nucleic acid present can be determined. In either a quantitative or a qualitative method, it is important to first ascertain that the quality control of the assay is ensured by the observation of the presence of the IC sequence which has been taken through at least some of the same steps as the target nucleic acid. For example, in nucleic acid-based assay methods which involve amplification, an amplification of the IC sequence in the absence of amplification of the target sequence will then be evidence of a correct negative result and the amplification of both sequences a correct positive result. No amplification of either the IC sequence or the target sequence might well indicate technical failure or some problem with the assay conditions, e.g. the presence of inhibitors.

In a qualitative nucleic acid-based assay involving amplification the amplification of the target sequence in the absence of amplification of the IC nucleic acid will not normally be regarded as a concern and such a sample would be noted as a correct positive result. However, it should be noted that in a quantitative assay involving the use of a “pseudo-ideal” or “near-ideal” IC sequence, the amplification of the target sequence in the absence of the amplification of the IC sequence is likely to indicate that the target sequence was present in such high quantities in the sample that the amplification of the IC sequence was, in effect, competed out. Competition of the IC nucleic acid and target nucleic acid for the same primers in fact provides the basis of one of the best methods of quantitation of the assay, i.e. by so-called “competitive PCR”.

The methods of the present invention as described above involving a “pseudo-ideal” or a “near-ideal” IC sequence are well suited to competitive PCR as the IC nucleic acid and the target nucleic acid may be amplified by the same primers. Thus the amount of amplification which will occur will be a function of the concentration of nucleic acid molecules present in the original sample. If the IC nucleic acid is present at a higher concentration than the target nucleic acid then this will be subject to more amplification and vice versa. When the ratio of IC nucleic acid to target nucleic acid is equal, i.e. 1:1, then one can expect the amount of amplification of both species to also be equal.

This provides a convenient way of quantitating the results. For example a series of samples can be set up containing various dilutions of a known amount of the IC nucleic acid and unknown amounts of the target nucleic acid. Analysis of the amplification products should reveal the concentration of IC nucleic acid at which the amplification of both species is roughly equal and the quantity of target nucleic acid present in the original sample can be extrapolated by analysing the amount of amplification products present at the various dilutions and drawing up standard curves. Quantitative competitive PCR is a standard procedure and is described for example in the review by Zimmerman et al., supra.

Relative quantitation can also be carried out using a series of dilutions containing constant unknown amounts of IC nucleic acid and varying known concentrations of the target nucleic acid. Clearly however the most likely scenario in any assay will be that it is the concentration of IC nucleic acid rather than target nucleic acid which is known and thus the first described method is likely to be more appropriate.

Any other suitable method of quantitation can be used to analyse the products of the nucleic acid-based assay, e.g. the amplification products. For example, the TaqMan system can be used where the accumulation of PCR products are measured in “real time” by release of two fluorescent dyes, one for the wild type and one for the IC targets (Heid et al., 1996, 6: 986-94, Tremmel et. al., 1999, Tissue Antigens, 54: 508-16).

The methods and uses as described above can be used in a number of different technical fields, for example in the fields of analysis and diagnosis. Most preferably the methods and uses can be used for qualitative or quantitative analysis of nucleic acids or for diagnosis.

In a yet further aspect, the present invention provides a kit for carrying out the methods or uses of the invention which comprises non-viable particles containing or comprising an appropriate IC nucleic acid as described and defined above.

Optionally said kits may further comprise one or more reagents selected from reagents suitable for nucleic acid amplification, such as appropriate primers or amplification probes (in the case of LCR) designed to be compatible with the IC nucleic acid in question and/or the target nucleic acid to be analysed and which may optionally be labelled, nucleotides (a proportion of which may be labelled), DNA polymerases, probes or other entities which can hybridise to the target nucleic acid and/or the IC nucleic acid and give rise directly or indirectly to a detectable signal, e.g. are optionally labelled, etc.

The invention will now be described in more detail in the following non-limiting Examples with reference to the following figures in which:

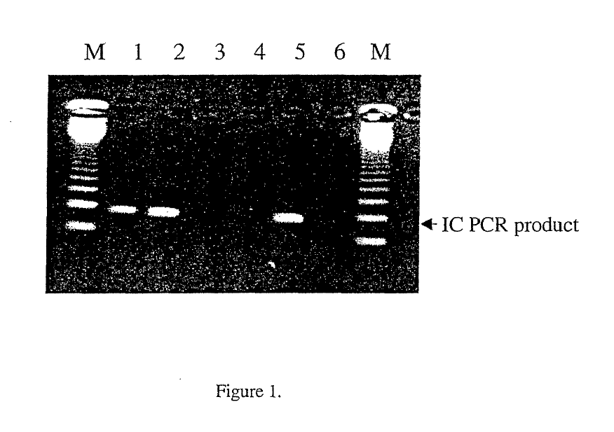

FIG. 1 shows an agarose gel with PCR products from nucleic acid encapsulated in POPC/DDAB liposomes. Lanes M, molecular size standard (123-bp ladder, Life Technologies); lane 1, liposomes with IC prepared without extrusion; lane 2, liposomes with IC prepared with extrusion; lane 3, procedure contamination control—liposomes prepared without DNA; lane 4, nuclease control—IC nucleic acid solution treated with DNase I and Exonuclease III; lane 5, procedure positive control—IC nucleic acid solution; lane 6, non-template PCR control;

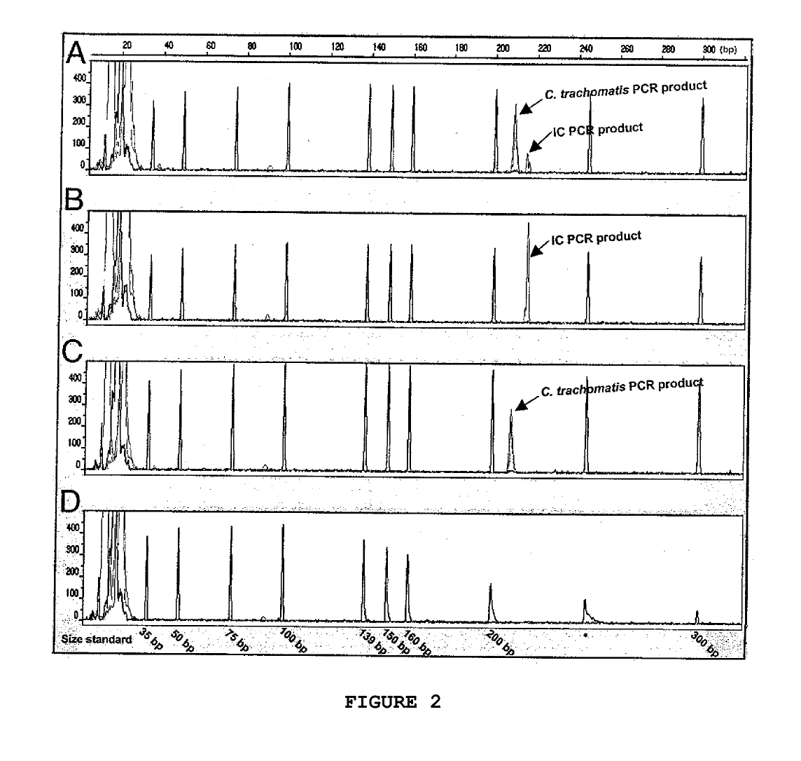

FIG. 2 shows the electropherogram after capillary gel electrophoresis of C. trachomatis PCR product (207 bp) and IC PCR product (216 bp) from nucleic acid from cultured C. trachomatis and/or from POPC/DDAB liposome/IC DNA complex. The size of the PCR products is interpolated from the internal size standard (GenScan-500 TAMRA, Applied Biosystems).

A. Cell culture of C. trachomatis spiked with POPC/DDAB liposomes containing IC nucleic acid.
B. POPC/DDAB liposomes containing IC nucleic acid without C. trachomatis.
C. Cell culture of C. trachomatis without spiking.
D. Non-template PCR control;

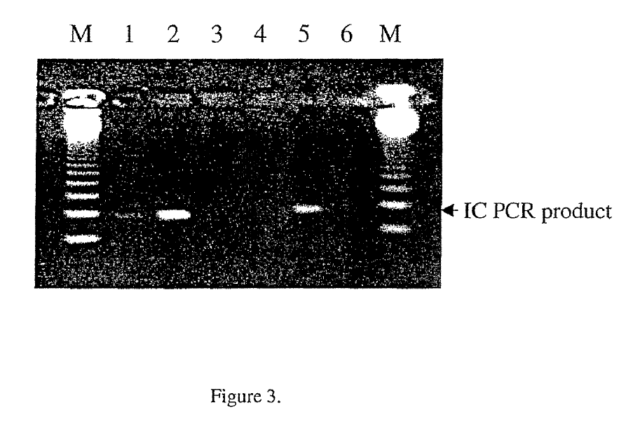

FIG. 3 shows agarose gel with PCR products from nucleic acid encapsulated in DOTAP/DOPE liposomes coated with polyethylene glycol. Lanes M, molecular size standard (123-bp ladder, Life Technologies); lane 1, liposomes with IC prepared without extrusion; lane 2, liposomes with IC prepared with extrusion; lane 3, procedure contamination control—liposomes prepared without DNA: lane 4, nuclease control—IC nucleic acid solution treated with DNase I and Exonuclease III; lane 5, procedure positive control—IC nucleic acid solution; lane 6, non-template PCR control.

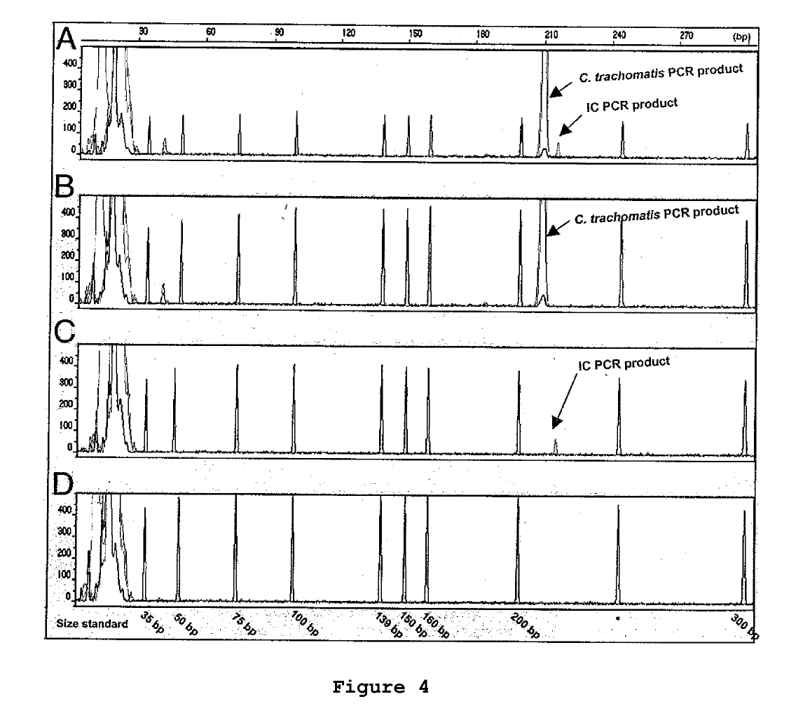

FIG. 4 shows the electropherogram after capillary gel electrophoresis of C. trachomatis PCR products (207 bp) and IC PCR products (216 bp) from nucleic acid from a urine specimen spiked with cultured C. trachomatis and/or POPC/DDAB liposome/IC DNA/Blue Dextran complex. The size of the PCR products is interpolated from the internal size standard (GenScan-500 TAMRA, Applied Biosystems).
A. Nucleic acid prepared from urine specimens spiked with cultured C. trachomatis and POPC/DDAB liposome/IC DNA/Blue Dextran complex.
B. Nucleic acid prepared from urine specimens spiked with cultured C. trachomatis.
C. Nucleic acid prepared from urine specimens spiked with POPC/DDAB liposome/IC DNA/Blue Dextran complex.
D. Negative control. Urine specimens without spiking.

### EXAMPLE 1

### Detection of IC Nucleic Acid Entrapped in Liposomes

### Production of the IC Nucleic Acid

A 216 bp segment of the phage M13 genome was chosen as an IC sequence and was amplified by use of published primers (Berg and Olaisen 1994, Biotechniques, 17: 896-901):

| LacL: | |
|---|---|
| 5′-GGCGAAAGGGGGATGTGC-3′ | SEQ ID NO: 1 |
| LacH: | |
| 5′-(FAM)-CGGCTCGTATGTTGTGTGGAAT-3′ | SEQ ID NO: 2 |

In the PCR production of the IC nucleic acid 1 ng M13mp18 DNA (Pharmacia) was used as template DNA followed by a 30 cycle PCR using the same conditions as published for the LacL/LacH primers. The resulting PCR product was purified by gelfiltration (Sephacryl S300, Pharmacia). Due to the FAM fluorescent modification of the LacH primer, the PCR product can be detected in an ABI Prizm 310 Genetic Analyzer by capillary gel electrophoresis and analysis by Genescan software.

Endpoint titration with subsequent PCR revealed the amount of the DNA in the purified IC solution.

### Preparation of the Liposomes and Encapsulation of the IC Nucleic Acid

Liposomes containing the lipids POPC and DDAB in a ratio of 97.5:2.5 are prepared by the freezing/thawing procedure as described by Monnard et al. (BBA, 1329: 39-50, 1997). The lipids are dissolved in chloroform and the solvent subsequently removed by evaporation followed by an overnight drying under high vacuum. The dried lipids are dispersed in a buffer solution (50 mM Tris, pH 8.0) and sonicated for 10 minutes in a bath sonicator. Then 10 μg of the IC nucleic acid to be encapsulated is added and the final concentration of lipids adjusted to 120 mM. The dispersion is treated by freeze/thawing 10 times. After freezing in liquid nitrogen, the samples are thawed for 15 minutes at room temperature. Before extrusion, the liposome dispersion is diluted to a lipid concentration of 40 mM using 50 mM Tris (pH 8.0) and then forced 10 times through two stacked polycarbonate filters with pore sizes of 400 nm in diameter (for extrusion a Liposofast from Avestin Inc. is used). The extruded liposomes are loaded on a ‘spin column’ (Bio-Gel A-15 m, previously equilibrated with the appropriate buffer, pH 8.0) and centrifuged at 165×g for 2 minutes. Usually 22-24 eluates of about 50 μl each are collected: the fractions 2-7 are usually turbid, the others showed no clearly visible turbidity, indicating that they contain no significant number of liposomes.

### Lysis of the Liposome/DNA Complex

Isolation of DNA from the suspension of liposomes with entrapped IC was done by use of a Qiagen DNA mini kit according to a procedure described by the manufacturer. Briefly, after lysis of the liposomes in the presence of detergents, nucleic acids were bound to a silica-gel membrane, washed and finally eluted in TE buffer.

### Analysis of Isolated IC DNA

The IC DNA isolated from the liposomes was analysed in a PCR reaction that included the LacL/LacH primers. The resulting PCR product was analysed after agarose gel electrophoresis and EtBr staining with visualisation under UV light.

### EXAMPLE 2

### Use of the IC for Quality Assurance in an Assay for Detection of Chlamydia Trachomatis

The following PCR primers were used in an assay for detection of Chlamydia trachomatis, such primers being slightly modified at the 5′ ends compared to the published primers (Loeffelholz et al. 1992, J. Clinical Microbiology, 30, 2847-51):

CP24
SEQ ID NO: 3
5′-GGGATTCCTGTAACAACAAGTCAGG-3′
CP27
SEQ ID NO: 4
5′-(ROX)-CCTCTTCCCCAGAACAATAAGAACAC-3′

These primers define an amplification product containing 207 bp of the C. trachomatis cryptic plasmid.

The production of the IC and the entrapment of it into liposomes was done as described in the above example 1.

Isolation of DNA from an In Vivo Sample Containing C. Trachomatis Spiked with Entrapped IC

A cell suspension of a cultured C. trachomatis L2 strain was spiked with liposomes/IC DNA complex prepared as described above. Chlamydia and IC DNA was prepared from the cells/liposomes by using a Qiagen DNA mini kit according to a procedure described by the manufacturer.

### PCR Analysis of the DNA Solution

The purified Chlamydia and IC DNA was analysed in a multiplex PCR including both of the above primer sets. The 35 cycles PCR was performed by use of Amplitaq Gold (Roche) as described by the manufacturer of the enzyme. Due to different fluorescence labelling of the Chlamydia and IC PCR products they were distinguishable in an ABI Prizm 310 Genetic Analyzer after capillary gel electrophoresis and analysis by Genescan software.

### EXAMPLE 3

### Preparation of Cationic Liposomes Coated with Polyethylene Glycol and Encapsulating an IC Nucleic Acid

The liposomes encapsulating an IC nucleic acid are prepared as described in Example 1. In this case however the liposomes contain the cationic lipid DOTAP [1,2-dioleoyloxy-3-(trimethylammonium)propane], the neutral lipid DOPE [1,2,dioleoyl-3-sn-phosphatidylethanolamine] and PEG-PE [N-(ω-methoxypoly(oxyethylene)oxycarbonyl)-DSPE] at a molar ratio 25:25:3 and extrusion was carried out through 50 nm pore filters [DSPE=1,2-distearoyl-3-sn-phosphatidylethanolamine].

### EXAMPLE 4

### Detection of IC Nucleic Acid Entrapped in Liposomes

### Production of the IC Nucleic Acid

A 216 bp segment of the phage M13 genome was chosen as an IC sequence and was amplified by use of published primers (Berg and Olaisen 1994, Biotechniques, 17: 896-901):

| LacL: | |
|---|---|
| 5′-GGCGAAAGGGGGATGTGC-3′ | SEQ ID NO: 1 |
| LacH: | |
| 5′-(FAM)-CGGCTCGTATGTTGTGTGGAAT-3′ | SEQ ID NO: 2 |

In the PCR production of the IC nucleic acid 1 ng M13mp18 DNA (Pharmacia) was used as template DNA followed by a 30 cycle PCR using the same conditions as published for the LacL/LacH primers. The resulting PCR product was purified by gelfiltration (Sephacryl S300, Pharmacia). The resulting PCR product was purified by gelfiltration (Sephacryl S300, Pharmacia). Endpoint titration with subsequent PCR revealed the amount of the DNA in the purified IC solution.

Due to the FAM fluorescent modification of the LacH primer, the Lac PCR product can be detected in an ABI Prizm 310 Genetic Analyzer by capillary gel electrophoresis and analysis by Genescan software.

### Preparation of the Liposomes and Encapsulation of the IC Nucleic Acid

Liposomes containing the lipids POPC (1-palmitoyl-2-oleoyl-sn-glycero-3-phosphocholine) and DDAB (didodecyldimethylammonium bromide) in a ratio of 97.5:2.5 were prepared by the freezing/thawing procedure as described by Monnard et al. (BBA, 1997, 1329: 39-50). The lipids were dissolved in chloroform and the solvent subsequently removed by evaporation followed by an overnight drying under high vacuum. The dried lipids were dispersed in a buffer solution (50 mM Tris, pH 8.0) and sonicated for 10 minutes in a bath sonicator. Then the IC nucleic acid to be encapsulated was added and the final concentration of the nucleic acid and lipids adjusted to 2.3 pM and 120 mM, respectively. The dispersion was treated by freeze/thawing 10 times. After freezing in liquid nitrogen, the sample was thawed for 15 minutes at room temperature. Before extrusion, the liposome dispersion was diluted to a lipid concentration of 40 mM using 50 mM Tris (pH 8.0). Extrusion was then carried out by forcing the liposome dispersion 10 times through two stacked polycarbonate filters with pore sizes of 400 nm in diameter (for extrusion a Liposofast from Avestin Inc. was used). After extrusion, the non-encapsulated nucleic acid was digested by a combined DNaseI and ExonucleaseIII treatment as described in Monnard et al., supra.

### Lysis of the Liposome/DNA Complex

Isolation of DNA from the suspension of liposomes with entrapped IC was done by use of a QIAamp DNA mini kit (Qiagen GmbH) according to a procedure described by the manufacturer. Briefly, after lysis of the liposomes in the presence of detergent, nucleic acid was bound to silica-gel membrane, washed and finally eluted in TE-buffer.

### Analysis of Isolated DNA

The IC DNA isolated from the liposomes was amplified in a PCR that included the LacL/LacH primers. The resulting PCR product was analysed after agarose gel electrophoresis and Ethidium bromide staining with visualisation under UV light (FIG. 1).

FIG. 1 shows an agarose gel with PCR products from nucleic acid encapsulated in POPC/DDAB liposomes. Seemingly, the extrusion through two polycarbonate filters enhances the entrapment efficiency of nucleic acid in the particles. Further, the results of the control experiments demonstrate that the IC nucleic acid was present in the core region of the liposomes and was protected from the nuclease digestion. Lanes M, molecular size standard (123-bp ladder, Life Technologies); lane 1, liposomes with IC prepared without extrusion; lane 2, liposomes with IC prepared with extrusion; lane 3, procedure contamination control—liposomes prepared without DNA; lane 4, nuclease control—IC nucleic acid solution treated with DNase I and Exonuclease III; lane 5, procedure positive control—IC nucleic acid solution; lane 6, non-template PCR control

### EXAMPLE 5

### Use of the Liposome/IC Complex for Quality Assurance in an Assay for Detection of Chlamydia trachomatis

The following PCR primers were used in an assay for detection of Chlamydia trachomatis, such primers being slightly modified with fluorescent dyes compared to the published primers (Loeffelholz et al. 1992, J. Clin. Microbiol., 30: 2847-51):

CP24
SEQ ID NO: 5
5′-(FAM)-GGGATTCC-(T-ROX)-GTAACAACAAGTCAGG-3′
CP27
SEQ ID NO: 6
5′-CCTCTTCCCCAGAACAATAAGAACAC-3′

(FAM and ROX are different fluorophores, green and red, respectively).

These primers define an amplification product containing 207 bp of the C. trachomatis cryptic plasmid.

The production of the IC and the entrapment of it into liposomes was done as described in the above Example 4.

Isolation of DNA from an In Vivo Sample Containing C. trachomatis Spiked with Entrapped IC

A cell suspension of a cultured C. trachomatis L2 strain was spiked with liposomes/IC DNA complex prepared as described above. Chlamydia and IC DNA was prepared from the cells/liposomes by using a QIAamp DNA mini kit (Qiagen GmbH) according to a procedure described by the manufacturer.

### PCR Analysis of the DNA Solution

The purified Chlamydia and IC DNA was analysed in a multiplex PCR including both of the above primer sets. The 25 cycles PCR was performed by use of Amplitaq Gold (Roche) as described by the manufacturer of the enzyme. Due to the different fluorescent labelling of the Chlamydia and Lac PCR products they were distinguishable in an ABI Prizm 310 Genetic Analyzer after capillary gel electrophoresis and analysis by Genescan software (FIG. 2).

### EXAMPLE 6

### Preparation of Cationic Liposomes Coated with Polyethylene Glycol and Encapsulation of an IC Nucleic Acid

The liposomes encapsulating an IC nucleic acid was prepared as described in Example 4. In this case however the liposomes contained the cationic lipid DOTAP [1,2-dioleoyloxy-3-(trimethylammonium)propane], the neutral lipid DOPE (1,2-dioleoyl-3-sn-phosphatidyl-ethanolamine) and PEG-PE [N-(ω-methoxypoly(oxyethylene)-oxycarbonyl)DSPE] at a molar ratio 25:25:3 [DSPE=1,2-distearoyl-3-sn-phosphatidylethanolamine] as described by Meyer et al, (JBC 1998, 273(25):15621-27). The PCR product from the released IC nucleic acid was analysed after agarose gel electrophoresis (FIG. 3).

FIG. 3 shows agarose gel with PCR products from nucleic acid encapsulated in DOTAP/DOPE liposomes coated with polyethylene glycol. As for the case with the POPC/DDAB liposomes described in Example 4, extrusion seems to enhance the entrapment efficiency of the nucleic acid into the particles. However, on comparing the results of Example 4 with the present example, no differences in the performance of the two liposome systems were observed. Lanes M, molecular size standard (123-bp ladder, Life Technologies); lane 1, liposomes with IC prepared without extrusion; lane 2, liposomes with IC prepared with extrusion; lane 3, procedure contamination control—liposomes prepared without DNA: lane 4, nuclease control—IC nucleic acid solution treated with DNase I and Exonuclease III; lane 5, procedure positive control—IC nucleic acid solution; lane 6, non-template PCR control.

### EXAMPLE 7

### Generation of Liposome/IC DNA/Blue Dextran Complex with Higher Density Allowing Sedimentation by Centrifugation and their Use in a Chlamydia trachomatis PCR Assay

The Lac PCR product used as IC nucleic acid was made as described in Example 4.

The preparation of the liposomes/IC complex was done as described in Example 4 with the following modification; Blue Dextran (Pharmacia) was added to the POPC/DDAB liposome dispersion together with the IC subsequent to the sonication. The concentration of the polysaccharide and the nucleic acid was adjusted to 75 nM and 23 pM, respectively. The subsequent freeze/thawing, extrusion and nuclease treatment was done as described in Example 4.

Isolation of DNA from a Urine Sample Containing C. Trachomatis Spiked with Liposome/IC DNA/Blue Dextran Complex

A urine sample collected from a healthy donor was spiked with a cell suspension of a cultured C. trachomatis L2 strain as well as with the liposome/IC DNA/Blue Dextran complex. Generation of a crude DNA lysate from the sample was done by use of the reagents provided in the commercially available COBAS AMPLICOR™ CT/NG assay (Roche Molecular Systems Inc. Diagnostics, Brachburg, N.J., USA) kit according to a procedure described by the manufacturer. Briefly, 500 μl of the urine specimen was diluted by 500 μl of the CT/NG Urine Wash solution, incubated at 37° C. followed by centrifugation at 12.500×g for 5 minutes. The cell/liposome pellet was re-suspended in 250 μl of the CT/NG LYS solution followed by 15 minutes incubation at room temperature. After addition of 250 μl of the CT/NG DIL solution, mixing and centrifugation at 12.500×g for 10 minutes, the crude DNA lysate was ready for PCR.

### PCR Analysis of the DNA Solution

An aliquot of the crude DNA lysate was added to a multiplex PCR reaction mixture including the two primer pairs listed in Example 4. After 30 cycles PCR that was performed as described, the PCR product was subjected to capillary gel electrophoresis and Genescan software analysis (FIG. 4).

1. A method of providing and using an internal control in a nucleic acid-based analysis of a sample, the method comprising
adding non-viable liposome particles, each non-viable liposome particle comprising an internal control (IC) nucleic acid, to a sample in which a target nucleic acid is contained within a target entity
wherein the internal control nucleic acid can be distinguished from the target nucleic acid, and
wherein said non-viable liposome particles are designed or modified such that said non-viable liposome particles have a density, weight, or size which is sufficiently similar to the target entity to allow co-separation in a density, weight, or size based separation method, and such that said non-viable liposome particles and said target entities are able to undergo all of the same treatment steps;
co-separating the target entity and the non-viable liposome particles from the sample; and
using the IC nucleic acid as an internal control in an analysis of the target nucleic acid.

2. The method of claim 1 wherein said design or modification comprises design or modification of all or some of the lipids comprising said non-viable liposome particles.

3. The method of claim 1 wherein said non-viable liposome particles have been modified so that the density, weight, or size of said non-viable liposome particles is increased.

4. The method of claim 3 wherein said non-viable liposome particles have been modified to incorporate polysaccharides or to fill a central core of said non-viable liposome particles with a dense solution.

5. The method of claim 4 wherein said polysaccharides are Blue Dextran or Dextran sulphate.

6. The method of claim 1 wherein said non-viable liposome particles are disrupted under the same conditions which will disrupt the target entity.

7. The method of claim 1 wherein said non-viable liposome particles are designed or modified such that said non-viable liposome particles have a stability under assay and sample conditions which is the same as the target entity, or which is similar enough to allow said IC nucleic acid to undergo the same treatment steps as the target nucleic acid contained within the target entity.

8. The method of claim 1 wherein the stability of said non-viable liposome particles is modified.

9. The method of claim 8 wherein the stability of said non-viable liposome particles is increased.

10. The method of claim 9 wherein said non-viable liposome particles are coated with inert hydrophilic polymers or proteins derived from thermally stable bacteria, or are modified using lipids derived from thermophilic bacteria, or wherein said non-viable liposome particles are not modified by cross-linked proteins.

11. The method of claim 10 wherein the hydrophilic polymer is PEG or the protein derived from thermally stable bacteria is an S-layer protein.

12. The method of claim 1 wherein said non-viable liposome particles comprise a proportion of phospholipids which are phospholipid derivatives of polyethylene glycol.

13. The method of claim 1 wherein said non-viable liposome particles are cationic liposomes.

14. The method of claim 1 wherein said non-viable liposome particles comprise one or more of the neutral lipids POPe (1-palmitoyl-2-oleoyl-sn-glycero-3-phosphocholine), and DOPE (1,2-dioleoyl-3-sn-phoaphatidylethanolamine), and one or more of the positively charged lipids DDAB (dimethyldioctadecylammonium bromide), DOTAP (1,2-dioleoyloxy-3-(trimethylamnionium) propane), or DOSPA (2,3-dioleyloxy-N-[2 (spertnine-carboxamido) ethyl]-N,N-dimethyl-1-propanaminiumtrifluoroacetate).

15. The method of claim 1 wherein said target entity is a cell derived from multicellular or unicellular organisms.

16. The method of claim 1 wherein said target entity is a virus or bacteriophage.

17. The method of claim 1 wherein the IC nucleic acid is encapsulated within said non-viable liposome particles.

18. The method of claim 1 wherein the IC nucleic acid is a pseudo-ideal or a non-ideal IC nucleic acid.

19. The method of claim 1 wherein the IC nucleic acid is from 50 to 500 bases in length.

20. The method of claim 1 wherein the nucleic acid-based analysis is a technique which involves amplification of the target nucleic acid.

21. The method of claim 20 wherein the nucleic acid-based analysis is PCR, LCR, Gap-LCR, NASBA or TMA.

22. The method of claim 1 wherein the nucleic acid-based analysis is quantitative.

23. The method of claim 1 wherein said non-viable liposome particles are separated from the sample together with the target entity using centrifugation or sedimentation.

24. The method of claim 1, wherein the density, weight, and/or size of said non-viable liposome particles is modified.

25. A method of nucleic acid-based analysis wherein an internal control nucleic acid sequence is used as an internal control, comprising:
bringing a sample to be analyzed into contact with non-viable liposome particles,
wherein each non-viable liposome particle comprises an internal control (IC) nucleic acid,
wherein the sample comprises a target entity comprising a target nucleic acid,
wherein the internal control nucleic acid can be distinguished from the target nucleic acid, and
wherein the non-viable liposome particles are designed or modified such that the non-viable liposome particles have a density, weight, or size which is sufficiently similar to the target entity to allow co-separation in a density, weight, or size based separation method, and such that said non-viable liposome particles and said target entities are able to undergo all of the same treatment steps;
co-separating the target entity and the non-viable liposome particles from the sample; and
analyzing the target nucleic acid and the IC nucleic acid, such that analysis of the IC nucleic acid functions as an internal control for analysis of the target nucleic acid.

26. The method of claim 25, further comprising:
obtaining the sample to be analyzed; and
inducing the release of the target nucleic acid from the target entity and the release of the internal control nucleic acid from the liposome particles.

27. A method of providing and using an internal control in a nucleic acid-based analysis of a sample, the method comprising
adding non-viable liposome particles, each non-viable liposome particle comprising an internal control (IC) nucleic acid, to a sample in which a target nucleic acid is contained within a target entity
wherein the internal control nucleic acid can be distinguished from the target nucleic acid, and
wherein said non-viable liposome particles are designed or modified by introducing a surface protein found on the target entity or a molecule found on the target entity into or onto said non-viable liposome particles to allow said liposome particles to be selectively captured with the target entity, and such that said non-viable liposome particles and said target entities are able to undergo all of the same treatment steps;
co-separating the target entity and the non-viable liposome particles from the sample; and
using the IC nucleic acid as an internal control in an analysis of the target nucleic acid.

28. The method of claim 27 wherein said non-viable liposome particles are selectively captured by affinity separation or immunoseparation.

29. A method of nucleic acid-based analysis wherein an internal control nucleic acid sequence is used as an internal control, comprising:
bringing a sample to be analyzed into contact with non-viable liposome particles,
wherein each non-viable liposome particle comprises an internal control (IC) nucleic acid,
wherein the sample comprises a target entity comprising a target nucleic acid,
wherein the internal control nucleic acid can be distinguished from the target nucleic acid, and
wherein the non-viable liposome particles are designed or modified by introducing a surface protein found on the target entity or a molecule found on the target entity into or onto said non-viable liposome particles to allow said liposome particles to be selectively captured with the target entity, and such that said non-viable liposome particles and said target entities are able to undergo all of the same treatment steps;
co-separating the target entity and the non-viable liposome particles from the sample; and
analyzing the target nucleic acid and the IC nucleic acid, such that analysis of the IC nucleic acid functions as an internal control for analysis of the target nucleic acid.
